(19) United States
(12) Patent Application Publication
(10) Pub. No.: US 2013/0319404 A1
(43) Pub. Date: Dec. 5, 2013

(54) Liquid Droplet Spray Device

(71) Applicants: Amir Feriani, Auvernier (CH); Christian Tâche, Peseux (CH); Jean-Paul Sandoz, Cormondreche (CH); Cèdric Zaugg, Neutchatel (CH)

(72) Inventors: Amir Feriani, Auvernier (CH); Christian Tâche, Peseux (CH); Jean-Paul Sandoz, Cormondreche (CH); Cèdric Zaugg, Neutchatel (CH)

(73) Assignee: British American Tobacco (Investments) Limited, London (GB)

(21) Appl. No.: 13/883,702

(22) PCT Filed: Oct. 31, 2011
(86) PCT No.: PCT/EP11/69158

§ 371 Date: Aug. 22, 2013

(30) Foreign Application Priority Data
Nov. 8, 2010 (GB) 1018796.1
Mar. 4, 2011 (GB) 1103692.8

(51) Int. Cl. A61M 15/00; B05B 17/06

(52) U.S. Cl. CPC A61M 15/009; B05B 17/0615
USPC 128/200.14; 239/102.1; 239/102.2

(57) ABSTRACT

A liquid droplet spray device for ejecting a liquid as a spray of droplets has a cartridge (15) with a nozzle arrangement (13) to eject droplets, which is vibrated by a piezo element (23a) through the agency of an actuating element (23). A resonance chamber is disposed between an inner wall (15a) of the cartridge and the actuating element (23) such that the cartridge inner wall (15a) is separated by a gap having a length B from the actuating element (23), the cartridge inner wall (15a) being cap-shaped and extending along the side surfaces of the actuating element (23) and being frictionally attached to the side surfaces along a distance A. The resonance chamber (10) improves the efficiency of transmission of vibrations to the cartridge.

The present invention relates to a droplet spray device.

Such droplet spray devices are also sometimes called aerosol generators, nebulizers and the like. They normally contain a nozzle body on a support part, in particular, a nozzle body of a liquid droplet spray device which dispenses a liquid substance as a liquid droplet spray or from the device through the nozzles of the nozzle body. They further consist of an actuator based on a vibrating element which generally causes the liquid to vibrate, to be accelerated and expelled as droplets. They further consist of elements such as liquid space, liquid feed and fluid interface to a reservoir, a reservoir as well as electrical connections between the vibrating element and a corresponding electronic circuitry. Said elements may be contained in the aforementioned support part, in a further support part or they may be contained in a number of support parts. Said support part or parts and elements need to be manufactured and assembled with the actuator and the vibrating element. Said liquid may be for example an ambient fragrance, a perfume, an insecticide, a liquid pharmaceutical formulation, aqueous based liquids and flammable or combustible liquids.

Such nozzle bodies are sometimes called aperture plates, nozzle arrays, dosing aperture, orifice plate, vibratable membrane member, dosing aperture arrangement, aerosol generator and the like. Said terms are hence to be understood as being interchangeable throughout the present document.

Such nozzle bodies and droplet spray devices are well known. For example, see EP 1 129 741. This document describes a liquid droplet spray device having a top substrate formed of a main body and of a nozzle body. The nozzle body contains a nozzle array of liquid droplet outlet means allowing a liquid substance contained in the liquid droplet spray device to exit the device, in this case as a spray of droplets. The nozzle body is conventionally formed of a nozzle array made out of silicon, a polymer, a resin such as SU-8, Nickel, a metal alloy, Parylen, Duroplast or any suitable material or combination of these and other materials that allows for a sufficiently precise and cost-effective manufacturing of the outlet nozzle array. Beyond well-known silicon, metal and SU-8 resin micro-machining methods said nozzle array could also be produced by methods using tools made with silicon micro-machining and other known replication methods like LIGA (Lithography-Galvano forming), hot embossing, UV printing, polymer and powder micro-injection moulding, micro-EDM and similar advanced 3D micro-machining methods and suitable combination of methods using photolithography and micro-structuring of resins, silicon, metal and plastic.

U.S. Pat. No. 6,722,582 and EP 1 273 355 disclose such micro-machining methods.

PCT/EP2006/006059 shows a droplet spray device including the nozzle body, the support parts and the actuator containing the vibrating element as well as a general way of assembling such a device.

US 2004/0263567 and EP-A-1 604 701 show examples of various device configurations for which such a droplet spray device can be produced and needs to be assembled into in an efficient and cost-effective manner.

As can be seen from the cited prior art documents, all of them have a problem to assemble the actuator in a way which provides an efficient use of the ultrasonic energy delivered by the vibrating element, namely a piezoelectric element.

Another problem is that leak-tightness needs to be guaranteed for a variety of liquids. Leak-tightness normally implies rigid body construction and assembly of its components and long-term resistance of the components to sometimes aggressive solvents.

A further problem is represented by the need to disassemble the droplet spray device after one or several uses in order not to discard all parts after use, but to discard only one part and to keep the others for further use after cleaning for example or to disassemble some parts for cleaning them periodically and to reassemble them again for further use.

As can be understood by the person skilled in the art, these criteria can be highly contradictory in their requirements and effects. Also, as said before none of the prior art devices discussed above discloses on how to achieve these contradictory criteria in one device or a family of devices.

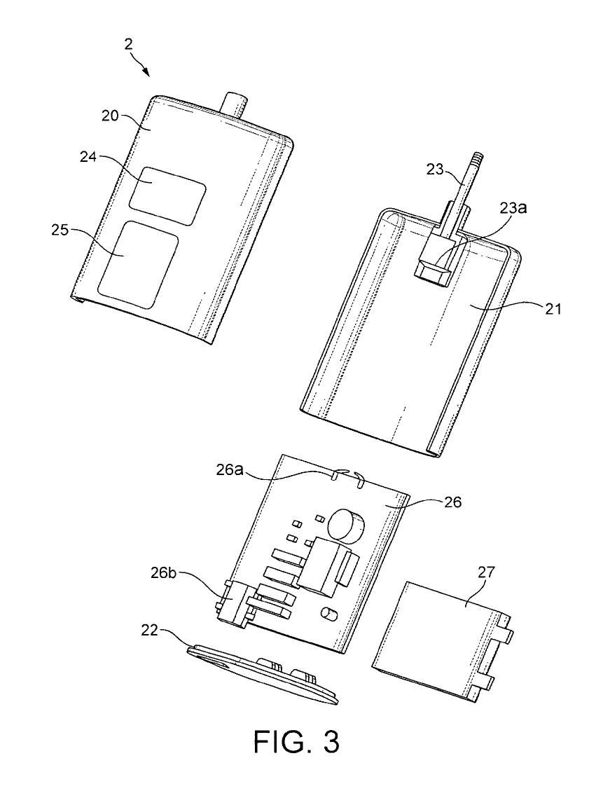

EP 2130611 describes a volatile liquid dispenser device for ejecting liquid as a spray of droplets. A piezoelectric element acts on the liquid so as to cause the liquid to undergo vibration by transmission of the ultrasound from the piezoelectric element to the liquid. This device is schematically shown in FIG. 3 of that document EP 2130611. An actuating membrane is positioned between a first substrate and a second substrate, and can be actuated, i.e. put into vibration, by way of a vibrating element suitably attached thereto.

The first substrate is provided with a space, for example a recessed portion, allowing to receive liquid that is to be expelled through ultrasound transmission from the vibrating element to the liquid. This space, once filled with liquid, thus constitutes a pressure chamber for ejecting the liquid contained therein. A liquid droplet outlet means is further provided to allow for ejection of the liquid there through. This liquid droplet outlet means generally is a spray head consisting of a perforated membrane having a nozzle array, in a manner well known in the art.

However, the device only partially addresses the above-mentioned problem relating to efficiency of ultrasonic sound transmission so as to allow for improved efficiency of a piezoelectric element for actuating the liquid to be expelled. It has been found that transmission efficiency is lost from the vibrating element to the actuating element and then to the liquid in the space.

If this ultrasonic energy transmission can be further improved, a reduction in power consumption can be achieved, which is important especially when using battery-powered devices.

The present invention concerns the construction of an innovative spray device fulfilling these objectives efficiently and in various embodiments which may be obtained in a relatively simple and inexpensive manner.

The innovative dispenser device is defined in the appended claims. Thanks to the construction of the innovative and inventive spray device according to the present invention an efficient device fulfilling these objectives in various embodiments may be obtained in a relatively simple and inexpensive manner.

Other features and advantages of the spray device according to the present invention will become clear from reading the following description, which is given solely by way of a non-limitative example with reference to the attached drawings in which:

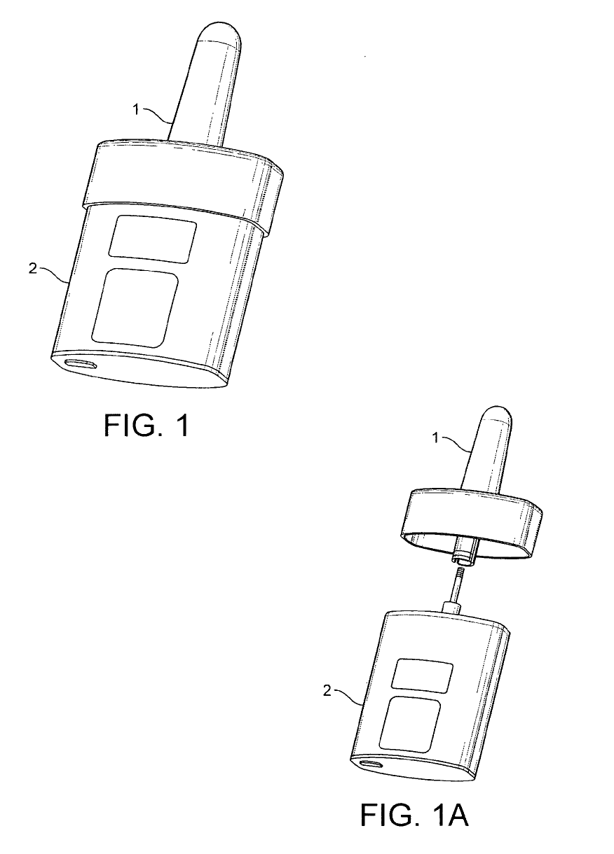

FIG. 1 shows an example of a nasal spray device according to the present invention;

FIG. 1A shows an exploded design of the nasal spray device of FIG. 1;

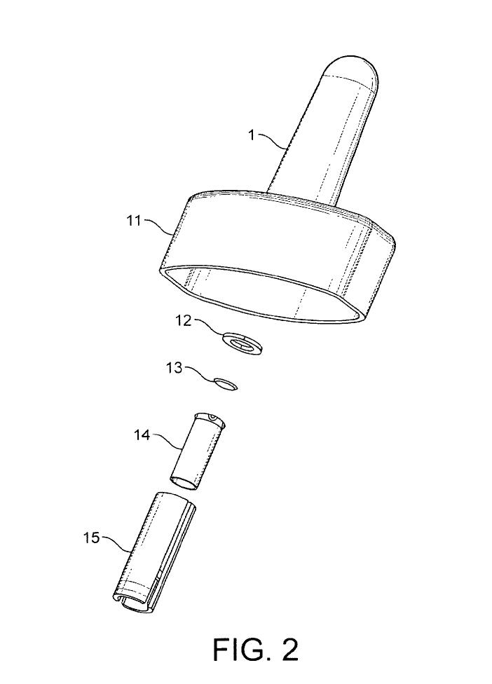

FIG. 2 shows an exploded design of a disposable part for a nasal spray device according to the present invention;

FIG. 3 shows in detail an exploded view of the spray device according to the present invention;

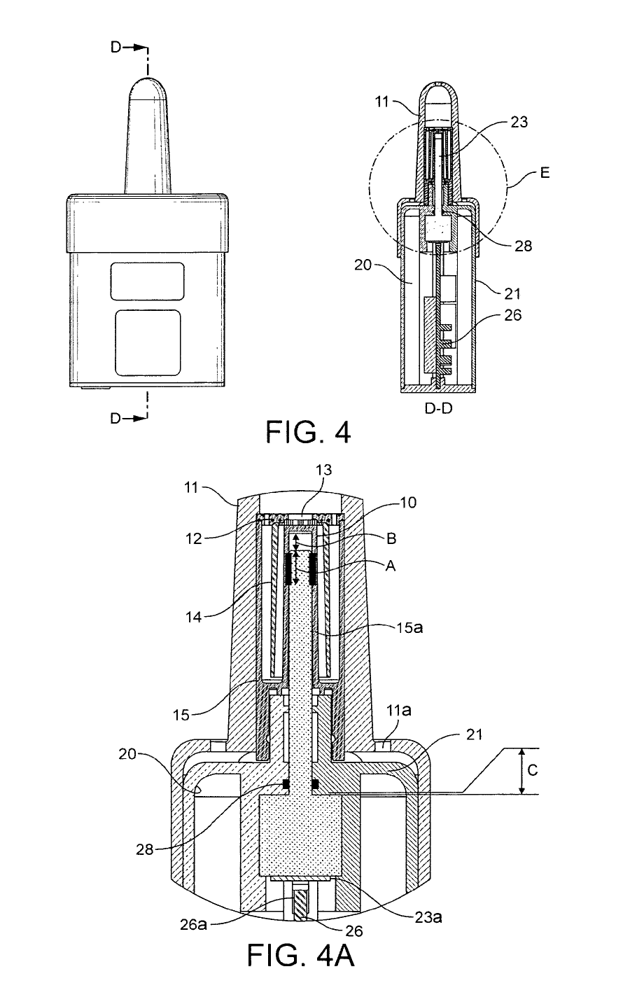

FIG. 4 shows a cross section view of the spray device with the disposable part according to the present invention;

FIG. 4A shows a zoom of the cross section view of the spray device of FIG. 4;

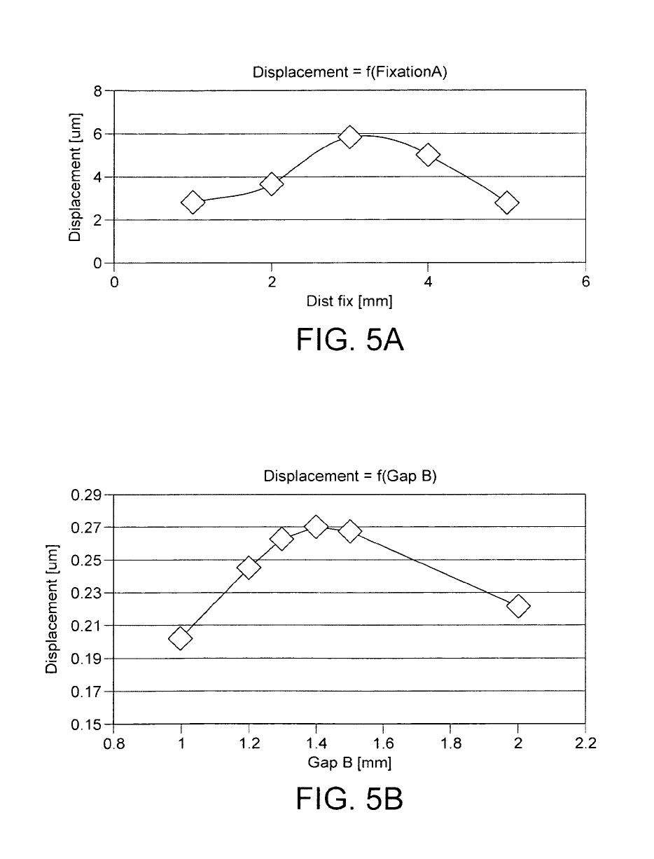

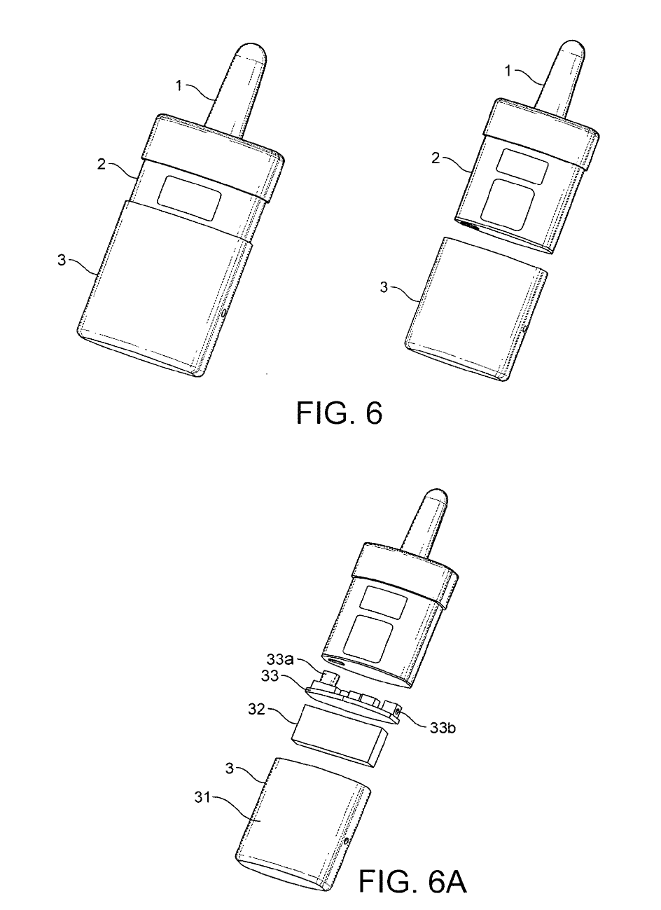

FIG. 5A shows a graph of the displacement of the actuating element vs. the fixation distance A of the spray device according to the present invention;

FIG. 5B shows a graph of the displacement of the actuating element vs. the length of gap B of the spray device according to the present invention;

FIG. 6 shows an exploded view of a spray device and a backup battery assembly according to the present invention;

FIG. 6A shows an exploded design of the backup battery assembly of FIG. 6;

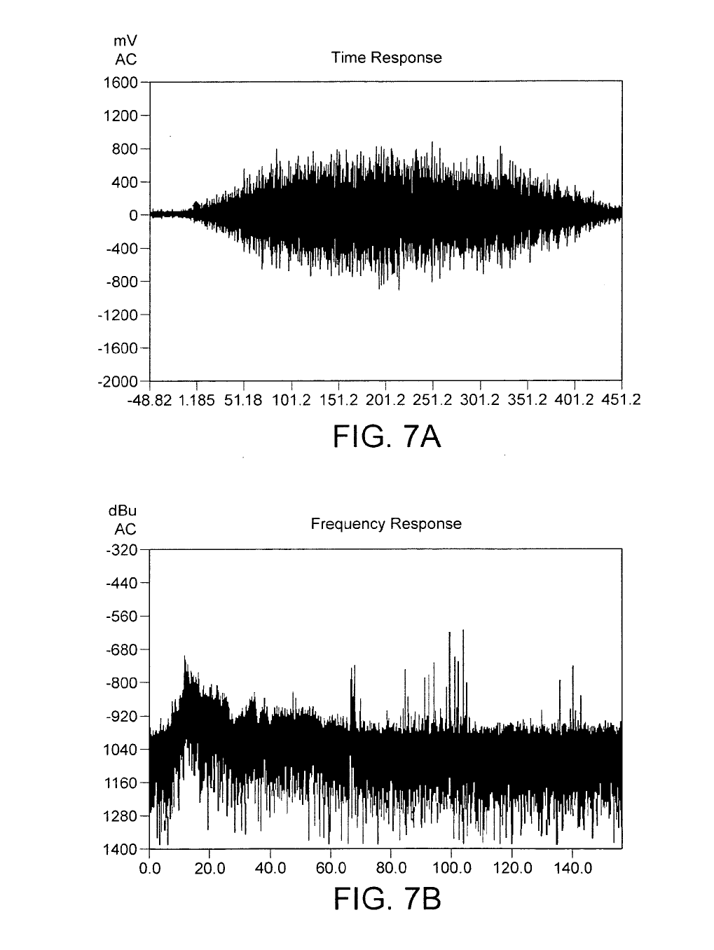

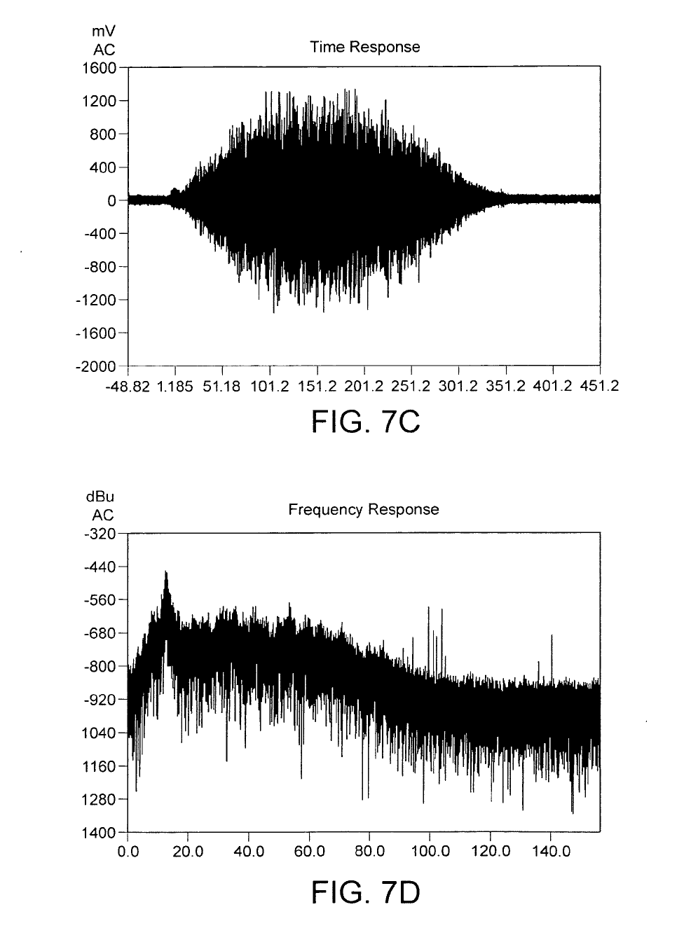

FIGS. 7a and 7b show a time domain response and a frequency response for a detected inhalation airflow of a person using a self-sensing spray device in the alternative embodiment; and

FIGS. 7c and 7d show in analogy the detected exhalation flow of the self-sensing spray device in the alternative embodiment.

The present invention thus concerns a liquid droplet spray device. The device may have a nasal-piece or a mouthpiece for delivery of the spray to the nose or mouth, depending on the liquid and function thereof used in the spray device. In the following examples, a nasal spray device will be described, but of course the present invention equally applies to a mouth spray device, as in principle the nasal-piece through which the spray is expelled can be simply substituted with a suitable mouthpiece.

FIG. 1 shows an example of a nasal spray device according to the present invention. In this example, the spray device consists of two parts, a first part 1 comprising a nasal-piece (or mouthpiece as the case may be), and a second, or fixed, part 2. First part 1 may be disposable. However, both parts 1 and 2 could be non-disposable. FIG. 1A shows a view of the disposable part 1 being separated from the fixed part 2.

FIG. 2 shows an exploded design of a disposable part for a nasal spray device according to the present invention. Disposable part 1 comprises nasal-piece 11 acting as liquid outlet means as liquid contained in the spray device will be expelled through the nasal-piece. As mentioned above, this may be a mouthpiece instead. Disposable part 1 further comprises a cartridge body 15, a capillary wall element 14, a nozzle body 13, and a cartridge cover 12, which are disposed inside nasal-piece 11. Capillary wall element 14 is conceived so as to slide into cartridge body 15. Cartridge body 15 may be cylindrically-shaped and is provided with a concentric cartridge inner wall 15a and is positioned over actuating element 23 to cover it. The top surface of cartridge inner wall 15a is in contact with nozzle body 13. Cartridge cover 12 is positioned above nozzle body 13 and on top of cartridge body 15 to close it.

FIG. 3 shows in detail an exploded view of the fixed part of the nasal spray device. Fixed part 2 comprises a front side section 20, a backside section 21, and a bottom side section 22. A PCB is provided between front side section 20 and backside section 21 and contains electronic control means 26 powered by a battery 27 suitably arranged in fixed part 2, for example between front side section 20 and the PCB as shown in FIG. 4 in more detail, and which will be discussed in more detail hereafter.

Advantageously, a connector 26b may be provided on PCB to allow for external communication to electronic control means 26. Connector 26b may be for example a USB connector allowing to program the electronic control means or to read out stored data therein. Also, battery 27 could be chargeable through such USB connector in a manner well known in the art of computer peripherals. An LCD screen 24 may be provided on front side section 20 for showing general information relating to the operation of the spray device such as information about the liquid to be sprayed, the duration of spraying, whether the device is on etc. The PCB further comprises connection means 26a for connection with an external element, in this case a vibrating element 23a, as will be explained later.

Further, a digitizer 25 may be also provided on front side section 20 to allow authorised access to the operation of the spray device. For example, if the liquid to be sprayed is a medication that may be harmful for others, only the person for whom the spray is intended can activate the spray device by way of digitizer 25.

This improves operation safety of the device.

Fixed part 2 also comprises a vibrating element 23a, for example a piezoelectric element, and an actuating element 23 that can be actuated by way of a vibrating element 23a. When vibrating element 23a is activated, by way of connections means 26a and electronic control means 26, the ultrasound energy generated by vibrating element 23a is transmitted to actuating element 23 and then to liquid present in nozzle body 13, thus causing the liquid to undergo vibrations and to be expelled as a spray of droplets.

Actuating element 23 is in a horn-shape. The horn shape may be convergent or step-shaped. In a preferred arrangement, actuating element 23 is a stepped horn having two sections. One section proximate to the vibrating element 23a may be a ¼ wavelength long while the second section being a ¾ wavelength long. Actuating element 23 is fixedly attached to vibrating element 23a, and this pair is arranged so as to be aligned with the nozzle body 13 of the spray device so as to allow for optimal use of the generated ultrasound energy.

Due to its horn-shape, actuating element 23 functions as a mechanical amplifier by transmitting the ultrasound generated by the vibrating element 23a towards the liquid in an efficient manner.

FIG. 4 shows a cross-section view along lines D-D of the spray device of FIG. 1, where FIG. 4A shows a zoom of the cross section view of the nasal spray device of FIG. 4.

As shown, an air outlet 11a may be provided in the nasal-piece to ensure correct ejection of liquid by compensating for possible differences in atmospheric pressure in and out of the nasal-piece. Nasal-piece 11 is fitted over cartridge body 15. The top surface of cartridge inner wall 15a is in contact with nozzle body 13 to allow for transmission of ultrasound from the vibrating element to the actuating element to cartridge inner wall 15a to liquid in nozzle body 13. As also shown, actuating element 23 is sandwiched between front side section 20 and backside section 22. A seal 28 may be provided to reduce ultrasonic wave losses and to ensure water tightness. In this example, cartridge inner wall 15a is in the shape of a cylinder and fits over horn-shaped actuating element 23. However, cartridge inner wall 15a does not touch the top surface of actuating element 23, but instead is separated therefrom by a distance B. The thus generated gap constitutes a resonance chamber 10 between actuating element 23 and nozzle body 13. This distance B can be obtained by a suitable design of cartridge inner wall 15a and actuating element 23. In this embodiment, cartridge inner wall 15a slides over actuating element, but frictionally contacts the latter until it is fixed in place, for example by having a slightly tapered end of horn-shaped actuating element 23. Both elements are designed so as to have a frictional contact along a distance A measured from the top surface of actuating element 23.

FIG. 5A shows a graph of the displacement of the actuating element with respect to the fixation distance A. FIG. 6B shows a graph of the displacement of the actuating element with respect to the length of the gap B. As can be seen, by varying these distances A and B, an increased displacement of the actuating element is obtained. This means that an increased transmission of ultrasound energy is obtained thus resulting in a more efficient transmission of ultrasound energy from vibrating element 23a to liquid in nozzle body 13.

Nozzle body 13 may comprise a perforated nozzle membrane having a plurality of outlet nozzles. It further comprises a space for containing liquid to be expelled, in a manner well known in the art, such as for example disclosed by co-pending application EP 2130611.

In fact, the main difference between the present spray device and that of the co-pending application lies in the arrangement of cartridge inner wall 15a with respect to actuating element 23. Cartridge inner wall 15a acts as a sealing element, similar to the sealing element in the above-mentioned co-pending application EP 2130611.

We have determined that, contrary to what might be expected, the ultrasound energy is more efficiently transmitted to liquid in the space when providing a resonance chamber 10 between actuating element 23 and cartridge inner wall 15a with the respect to fixation A.

Indeed, in this arrangement, actuating element 23 can be optimised to allow for efficient transmission by pairing it with vibrating element 23a. Correct pairing of these elements may be achieved by using impedance-matching technique, as may be readily understood by a skilled person. Further, cartridge inner wall 15a covers the top surface of actuating element 23, albeit by being separate therefrom by a distance B, so as to create the gap B constituting the resonance chamber 10, and extends along the side surface of actuating element 23 where cartridge inner wall 15a is frictionally attached to the side surface of actuating element 23 along a distance A. Distance A should be sufficiently long to ensure a correct ultrasound energy transmission from the actuating element/vibrating element pair to the cartridge inner wall 15a, but the distance A should not be too long to avoid the actuating element from vibrating correctly, i.e. it should not intervene with the deformation of the actuating element by constraining it longitudinally.

In fact, according to the present invention, the inventors have found that by providing resonance chamber 10, the vibration of actuating element 23 is amplified and transmitted more efficiently to cartridge inner wall 15a and thus to any liquid present in the space of nozzle body 13 of the liquid droplet spray device. In fact, if cartridge inner wall 15a touches the top surface of actuating element 23, as is the case in the above-referenced co-pending application EP 2130611, it was found that the displacement of the cartridge inner wall 15a is reduced due to increased necessary force to maintain cartridge inner wall 15a in close contact with the actuating element 23.

In this arrangement, actuating element 23 can be fixed into the fixed part 2 with a gap C to avoid ultrasonic transmission losses.

Thus, by designing the actuating element in such a manner that it is structured to optimise ultrasound transmission, a more efficient spray device can be obtained, which thus uses less power.

Further, thanks to the use of intermediate cartridge inner wall 15a, and by fixedly attaching the latter to the nasal-piece 11, thus enclosing and sealing the nozzle body 13 and its space containing liquid to be expelled, it is possible to make the part 1 disposable. In fact, disposable part 1 is a separable part that is self-contained and may include pre-filled liquid in the nozzle body, or not, as any liquid will not seep out due to the presence of capillary wall element 14.

Advantageously, this self-contained part may be designed to be disposable, thus allowing for simple replacement of liquid, and avoiding any clogging of the nozzles in the perforated membrane over time. The fixed part 2, the actuating membrane and the vibrating element may be on the contrary designed as non-disposable parts.

In a variant, an external reservoir, not shown, may be provided for feeding liquid from the reservoir to nozzle body 13, in a manner known as such, and also described for example in co-pending application EP 08 157 455.0. Suitable capillary liquid feeding means, not shown, are then required for transmitting the liquid from the external reservoir to space 5, in a manner well known to a skilled person. Nozzle body 13 may be designed to receive a predetermined quantity of liquid, such as a unit dose. Nozzle body 13 may instead, however, have an internal space that is designed sufficiently large to act as an internal reservoir thus avoiding the need of an external reservoir.

In such manner, a separate disposable cartridge can be obtained which may be fitted onto fixed part 2, thereby ensuring contact between cartridge inner wall 15a, forming part of disposable part 1, and actuating element 23, forming part of fixed part 2.

Thanks to the above-described embodiment of the dispensing device according to the present invention, ultrasound energy transmission can be improved, thus leading to improved fluidic performance of the present spray device. As capillarity characteristics may further be optimised too, a further increase in fluidic performance may be obtained.

As may be understood, this leads to a more efficient device, requiring less ultrasound energy for ejecting a similar amount of liquid as compared to the conventional device as disclosed by co-pending application EP 2130611, so that less energy is required to operate the device, which is of course an important aspect, not only in a battery-powered liquid droplet spray device, but in general.

This improved device may be used for respiratory devices. For example, an inhalation device may be provided by using the liquid droplet spray device in combination with a mouthpiece and a fluidic interface allowing for spraying of liquid into a person's respiratory airway.

In a similar manner, the present liquid droplet spray device may be used as a nasal spray, or a fresh-breath spray. Other applications, for example, use as an eye-cleansing spray, or even as a medical cigarette substitution device allowing for precise control of sprayed quantities of liquid into a person's respiratory airway.

To further ensure reliable operation of the spray device, a secondary battery may be provided to use as a backup battery when battery 27 is empty or does not have sufficient power left to allow for a correct operation. This may prevent malfunctioning when a user requires a dose of liquid to be sprayed, since the backup battery can be quickly brought into use.

FIG. 6 shows an exploded view of a spray device and a backup battery assembly according to the present invention.

FIG. 6A shows an exploded design of the backup battery assembly of FIG. 6.

Backup battery assembly 3 comprises a backup battery housing 31, a backup battery 32 and an electronic charger control means 33. An external power supply connector 33b may also be provided for charging battery 27 and possibly also backup battery 32. Electronic charger control means 33 is provided with a battery charge connector 33a for connection with electronic control means 26 of fixed part 2 shown in FIG. 3. As electronic control means 26 may comprise a USB connector for charging, then battery charge connector 33a is of course adapted to connect thereto.

In an alternative arrangement, a time-control electronic circuit may be provided allowing for a time-controlled release of liquid. For example, in an inhalation device, it may be important to avoid overdoses by locking out the user for a specific predetermined time between doses.

Advantageously, an inhalation detection system may be combined with the above application to allow for automatic spraying, or not, of liquid. Such inhalation detection system may be the same as described in co-pending application EP 2216100 in the name of the present application.

In short, and as explained in the aforementioned co-pending application, the spray device is provided with actuating element 23 which is a piezoelectric actuator that converts mechanical perturbations to electricity. The mechanical perturbations may be caused by the inhalation and exhalation of a person using the spray device as an inhalation device. The principle is to use the detection of the inhalation and exhalation pattern of a person using the respiratory treatment device. Indeed, when putting the mouthpiece into the mouth, a person will inhale and exhale. This inhalation/exhalation causes perturbations of actuating element 23, so that the inhalation and exhalation airflows of the person can be detected. By appropriate analysis of these inhalation and exhalation sequences, the substance to be administered can be expelled as a spray by the spray device at the appropriate time to allow for an efficient treatment, i.e. while the person is actually inhaling, and not exhaling.

FIGS. 7a and 7b show the time response and the frequency response for a detected inhalation of a person. By using an appropriate time-frequency analysis, the beginning and the end of the inhalation process can be clearly detected. By using, for example, a threshold detection additional to the above analysis, electronic control means (not shown) may be provided to trigger release of liquid for spraying into the person's mouth after detection of the beginning of the inhalation process.

FIGS. 7c and 7d show in analogy the exhalation process detected by actuating element 23a. Thus, this process is carried out in an analogous manner to the one described above for the inhalation process. As such, triggering of the spray device may be prevented during exhalation.

By using these detection methods, the inhalation can be differentiated from the exhalation. Indeed, as can be seen from FIGS. 7a to 7d, the inhalation and exhalation can be differentiated by an appropriate time-frequency analysis.

Having described now the preferred embodiments of this invention, it will be apparent to one of skill in the art that other embodiments incorporating its concept may be used. It is felt, therefore, that this invention should not be limited to the disclosed embodiments, but rather should be limited only by the scope of the appended claims.

1. A liquid droplet spray device for ejecting a liquid as a spray of droplets, the device comprising:
a first part having a nozzle body for containing said liquid, and having liquid outlet means for ejecting liquid from said device, said nozzle body being arranged proximate to said liquid outlet means such that said liquid may exit the device by traversing said liquid outlet means,
a second part arranged to receive and hold said first part,
a vibrating element arranged to actuate liquid substance in said nozzle body such that the liquid undergoes a vibration and forces the liquid through the outlet means thereby exiting said device as a liquid droplet spray,
said spray device further comprising an actuating element arranged between said vibrating element and said first substrate nozzle body, and
a cartridge body provided with a cartridge inner wall arranged between said actuating element and said nozzle body for sealing liquid in said first part,
said actuating element and said cartridge inner wall being arranged to transmit ultrasound energy generated by vibration of said vibrating element to liquid in said nozzle body, and
a resonance chamber positioned between said cartridge inner wall and said actuating element such that said cartridge inner wall is separated by a gap having a length B from said actuating element, and by said cartridge inner wall being cap-shaped and extending along the side surfaces of said actuating element and being frictionally attached to said side surfaces along a distance A.

2. The liquid droplet spray device according to claim 1, wherein said actuating element is horn-shaped or step-shaped.

3. The liquid droplet spray device according to claim 1, wherein said liquid outlet means, said nozzle body and said cartridge body together form a separable part of said spray device.

4. The liquid droplet spray device according to claim 3, wherein said separable part is disposable.

5. The liquid droplet spray device according to claim 3, wherein said nozzle body is pre-filled with liquid to be expelled, and said separable part constitutes a disposable liquid cartridge.

6. The liquid droplet spray device according to claim 1, wherein said vibrating element is a piezoelectric vibrator.

7. The liquid droplet spray device according to claim 1, further comprising a battery for powering said vibrating element.

8. The liquid droplet spray device according to claim 7, further comprising electronic control means for controlling the activation of said vibrating element.

9. The liquid droplet spray device according to claim 7, wherein said battery is rechargeable.

10. The liquid droplet spray device according to claim 9, further comprising a connector allowing for an external connection to said electronic control means and for recharging said battery.

11. An inhalation device comprising:
the liquid droplet spray device according to claim 1,
a mouthpiece, and
a fluidic interface.

12. The device according to claim 11, wherein said vibrating element is arranged to detect a breathing pattern of a user through said mouthpiece.

13. The device according to claim 11, further comprising a digit recognizer for allowing authorized operation of said inhalation device.

14. A nasal spray device comprising:
the liquid droplet spray device according to claim 1,
a nasal piece, and
a fluidic interface.

15. The nasal spray device according to claim 14, further comprising a digit recognizer for allowing authorized operation of said nasal spray device.
